(19) United States
(12) Patent Application Publication
(10) Pub. No.: US 2014/0171731 A1
(43) Pub. Date: Jun. 19, 2014

(54) SURGICAL MESH FOR PROLAPSE REPAIR

(71) Applicant: Kurian Thott, Stafford, VA (US)

(72) Inventor: Kurian Thott, Stafford, VA (US)

(21) Appl. No.: 13/715,758

(22) Filed: Dec. 14, 2012

(51) Int. Cl. A61F 2/00

(52) U.S. Cl. CPC A61F 2/0045
USPC 600/37

(57) ABSTRACT

A surgical mesh for prolapse repair is anchored into the anterior longitudinal ligament of the sacrum and then adjusted. On the sacral end of the mesh, synthetic anchors are placed into the anterior longitudinal ligament of the sacrum. Once the anchors are placed, the mesh that is placed through the anchors can be adjusted based on the need of prolapse repair. Once adjusted, the mesh would be locked into place with a locking peg. The mesh takes the suturing away from the most critical part of the procedure, thereby enhancing safety and improving operative times. Conventional mesh do not include such a sacral anchoring system and has no way to be adjusted after placement.

### BACKGROUND OF THE INVENTION

The present invention relates to surgical mesh and, more particularly, to a surgical mesh for prolapse repair that is anchored into the anterior longitudinal ligament of the sacrum and adjusted.

When performing surgical prolapse repair, one of the most critical areas is the anterior longitudinal ligament of the sacrum. Risks of this area include massive bleeding if the needle is placed incorrectly, or entrapment of the ureter. The other issue is the difficulty in adjustment of the mesh after sutures are placed.

Currently, mesh that is used for sacrocolpopexy is a flat piece of mesh that is needs to be sutured into the sacrum and then the vagina. This design risks bleeding and ureter entrapment. Older designs require suturing of the mesh into the ligament on the sacrum. These designs have significant risks and takes significant time.

As can be seen, there is a need for an improved surgical mesh for prolapse repair.

### SUMMARY OF THE INVENTION

In one aspect of the present invention, a surgical mesh for prolapse repair comprises a vaginal side; a sacral side; and a plurality of mesh anchor arms forming the sacral side; and a plurality of anchors, each having an anchor eyelet for threading one of the plurality of mesh anchor arms therethrough.

In another aspect of the present invention, a method for placing a surgical mesh in a patient comprises placing anchors of a surgical mesh into an anterior longitudinal ligament of a sacrum of the patient, the surgical mesh having a vaginal side, a sacral side, a plurality of mesh anchor arms forming the sacral side, and a plurality of the anchors, each having an anchor eyelet with one of the plurality of mesh anchor arms threaded therethrough; suturing the vaginal side of the surgical mesh into a vagina of the patient; adjusting the mesh anchor arms by sliding the mesh anchor arms through the anchor eyelet of the anchors; and securing the mesh anchor arms at a desired adjustment.

These and other features, aspects and advantages of the present invention will become better understood with reference to the following drawings, description and claims.

### BRIEF DESCRIPTION OF THE DRAWINGS

FIG. 1 is a perspective view of a surgical mesh, in use, according to an exemplary embodiment of the present invention;

FIG. 2 is a top view of the surgical mesh of FIG. 1; and

FIG. 3 is a side detail view of the surgical mesh of FIG. 1.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a surgical mesh for prolapse repair that is anchored into the anterior ligament of the sacrum and then adjusted. On the sacral end of the mesh, synthetic anchors are placed into the anterior longitudinal ligament of the sacrum. Once the anchors are placed, the mesh that is placed through the anchors can be adjusted based on the need of prolapse repair. Once adjusted, the mesh would be locked into place with a locking peg. The mesh takes the suturing away from the most critical part of the procedure, thereby enhancing safety and improving operative times. Conventional mesh do not include such a sacral anchoring system and has no way to be adjusted after placement.

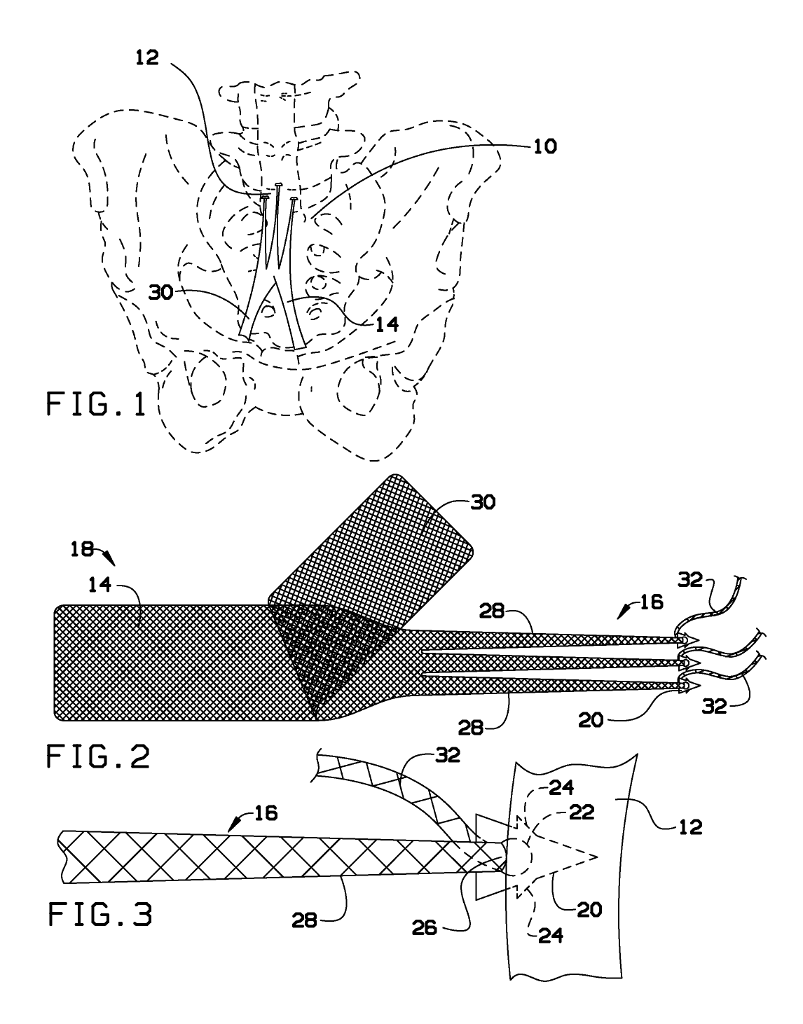

Referring now to FIGS. 1 through 3, a surgical mesh can be designed with a posterior flap 30 and an anterior flap 14 on a vaginal side 18 of the mesh. On a sacral side 16 of the mesh, a plurality of anchor arms 28, typically three anchor arms 28, can extend from the flaps 14, 30. Anchors 30 can be disposed at distal ends of each of the anchor arms 28. The anchors 20 can include an anchor eyelet 22 through which a mesh adjustment band 32 extends. The mesh adjustment bands 32 are formed integrally with the anchor arms 28 so that pulling on the mesh adjustment bands 32 causes the mesh band running through the anchor 26 to pull through the anchor eyelet 22, providing adjustment of the surgical mesh, as described in greater detail below. An anchor hook or screw 24 can be provided in the anchors 20 to help secure the anchors 20 in the anterior longitudinal ligament 12 of the sacrum 10.

The mesh of the present invention can be placed by first anchoring the anchors 20 in the anterior longitudinal ligament 12 of the sacrum 10. The vaginal side 18 of the mesh is sutured, as in traditional colpopexy. Then, the adjustable mesh bands 28 are pulled through the anchor eyelets 22 to adjust the mesh. The adjustable mesh bands 28 can then be locked in place through various mechanisms. For example, a peg (not shown) can be placed in the eyelet to prevent further movement of the adjustable mesh bands 28 therethrough. In some embodiments, pegs can be placed through the free mesh arm (mesh adjustment band 32) and then pushed into place.

The mesh can be placed either in an open or laparoscopic approach. The area around the sacrum can be prepped in the usual way. Once the ligament is identified, the anchors are placed, one at a time, into the ligament in a triangular or vertical manner. The vaginal portion of the mesh is then placed into the anterior and posterior aspects of the vagina and sutured into place. Once the mesh is secured into the vagina, the mesh arms on the sacral side can be adjusted and locked into place. Vaginal suturing can be done first as well or as the surgeon feels is adequate for the case.

Once placed, the need for suturing into the anterior longitudinal ligament of the sacrum will be unnecessary. This prevents the risk of injury to the middle sacral artery or to the ureter. The vaginal portion of the mesh is placed on either side of the dissected vagina and sutured in place as is traditionally performed. The mesh adjustment band can be trimmed, as needed, after locked into place.

Variable knit technology allows for a graft formed with meshes of differing densities. A soft, large pore construction in the single knit vaginal flaps allows for compliant organ support and host tissue ingrowth, while the sacral flap mesh is designed with incorporation of the anchors that are placed into the sacrum.

It should be understood, of course, that the foregoing relates to exemplary embodiments of the invention and that modifications may be made without departing from the spirit and scope of the invention as set forth in the following claims.

What is claimed is:

1. A surgical mesh for prolapse repair, comprising:
a vaginal side;
a sacral side;
a plurality of mesh anchor arms forming the sacral side; and
a plurality of anchors, each having an anchor eyelet for threading one of the plurality of mesh anchor arms therethrough.

2. The surgical mesh of claim 1, wherein the anchor includes an anchor hook for securing the anchor into an anterior longitudinal ligament of a sacrum of a patient.

3. The surgical mesh of claim 1, wherein the plurality of mesh anchor arms are three mesh anchor arms.

4. The surgical mesh of claim 1, wherein the vaginal side includes an anterior flap of mesh and a posterior flap of mesh.

5. A method for placing a surgical mesh in a patient, comprising:
placing anchors of a surgical mesh into an anterior longitudinal ligament of a sacrum of the patient, the surgical mesh having a vaginal side, a sacral side, a plurality of mesh anchor arms forming the sacral side, and a plurality of the anchors, each having an anchor eyelet with one of the plurality of mesh anchor arms threaded therethrough;
suturing the vaginal side of the surgical mesh into a vagina of the patient;
adjusting the mesh anchor arms by sliding the mesh anchor arms through the anchor eyelet of the anchors; and
securing the mesh anchor arms at a desired adjustment.

6. The method of claim 5, further comprising securing the anchor into an anterior longitudinal ligament of the sacrum of the patient with anchor hooks or screws formed into the anchors.

7. The surgical mesh of claim 5, wherein the plurality of mesh anchor arms are three mesh anchor arms.

8. The surgical mesh of claim 5, wherein the vaginal side includes an anterior flap of mesh and a posterior flap of mesh.
